(19) United States
(12) Patent Application Publication
(10) Pub. No.: US 2022/0013202 A1
(43) Pub. Date: Jan. 13, 2022

(54) METHODS, SYSTEMS, APPARATUSES AND DEVICES FOR FACILITATING MANAGEMENT OF PATIENT RECORDS AND TREATMENT

(71) Applicant: Nima Veiseh, Spring Lake, MI (US)

(72) Inventor: Nima Veiseh, Spring Lake, MI (US)

(21) Appl. No.: 17/372,358

(22) Filed: Jul. 9, 2021

Related U.S. Application Data
(60) Provisional application No. 63/049,707, filed on Jul. 9, 2020.

(51) Int. Cl. G16H 10/60; G16H 20/00; A61B 5/00

(52) U.S. Cl. CPC G16H 10/60; A61B 5/7264; A61B 5/6801; G16H 20/00

(57) ABSTRACT

Disclosed is a system for facilitating management of patient records. The system includes a communication device, a processing device, and a storage device. Further, the communication device may be configured for receiving a query from a user device, receiving a plurality of patient metadata from at least one patient metadata source, transmitting at least one request to at least one patient data source, receiving patient details corresponding to the at least one matching patient from the at least patient data source, and transmitting at least one treatment recommendation to the user device. Further, the storage device may be configured for storing the patient details. Further, the processing device may be configured for comparing the query with the plurality of patient metadata, identifying the at least one matching patient based on the comparing, analyzing the patient details, and generating the at least one treatment recommendation based on the analyzing.

The current application claims a priority to the U.S. Provisional Patent application Ser. No. 63/049,707 filed on Jul. 9, 2020.

### FIELD OF THE INVENTION

Generally, the present disclosure relates to the field of data processing and healthcare management. More specifically, the present disclosure relates to methods, systems, apparatuses and devices for facilitating management of patient records and treatment within a closed-loop ecosystem.

### BACKGROUND OF THE INVENTION

Existing techniques for facilitating management of patient and wellness records are deficient with regard to several aspects. For instance, current technologies do not recommend appropriate treatment options to users nor do they help to refine treatment options in a singular closed-loop ecosystem. This is novel in helping to facilitate the secure means of tracking, administering and refining care, and includes but is not limited to discovering, identifying, administrating and/or refining for treatments where government regulatory frameworks may not be fully defined. This security is provided by either standard existing methods or blockchain technology to create a closed loop ecosystem, where the data supply chain between physical goods and data are integrated, in what may be a feedback loop. The invention can also certify different levels of wellness care, across the entire ecosystem of health, wellness and/or integrative care.

Therefore, there is a need for improved methods, systems, apparatuses and devices for facilitating management of patient and wellness records that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a system for facilitating management of patient and wellness records (hereafter referred to as “patient records”). Further, the system may include a communication device, a processing device, and a storage device. Further, the communication device may be configured for receiving a query from a user device or an Application Programming Interface (API). Further, the communication device may be configured for receiving a plurality of patient metadata from at least one patient metadata source. Further, the communication device may be configured for transmitting at least one request to at least one patient data source. Further, the communication device may be configured for receiving one or more patient details corresponding to the at least one matching patient from the at least patient data source. Further, the communication device may be configured for transmitting at least one treatment recommendation to the user device. Further, the storage device may be communicatively coupled to the communication device. Further, the storage device may be configured for storing the one or more patient details. Further, the processing device may be communicatively coupled to each of the storage device and the communication device. Further, the processing device may be configured for comparing the query with the plurality of patient metadata. Further, the processing device may be configured for identifying the at least one matching patient based on the comparing. Further, the processing device may be configured for analyzing the one or more patient details. Further, the processing device may be configured for generating the at least one treatment recommendation based on the analyzing. Each of these devices may communicate with each other synchronously or asynchronously.

According to some embodiments, a method for facilitating management of patient records is disclosed. Accordingly, the method may include receiving, using a communication device, a query from a user device. Further, the method may include receiving, using the communication device, a plurality of patient metadata from at least one patient metadata source. Further, the method may include comparing, using a processing device, the query with the plurality of patient metadata. Further, the method may include identifying, using the processing device, at least one matching patient based on the comparing. Further, the method may include transmitting, using the communication device, at least one request to at least one patient data source. Further, the method may include receiving, using the communication device, one or more patient details corresponding to the at least one matching patient from the at least patient data source. Further, the method may include storing, using a storage device, the one or more patient details. Further, the method may include analyzing, using the processing device, the one or more patient details. Further, the method may include generating, using the processing device, the at least one treatment recommendation based on the analyzing. Further, the method may include transmitting, using the communication device, at least one treatment recommendation to the user device.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for facilitating management of patient records, in accordance with some embodiments.

FIG. 3 is a flowchart of a method for facilitating management of patient records, in accordance with some embodiments.

FIG. 4 is a flowchart of a method for facilitating management of patient records, in accordance with further embodiments.

FIG. 5 is a block diagram of a system for analyzing and reporting patient records for endocannabinoid healthcare, in accordance with some embodiments.

FIG. 6 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of facilitating management of patient records, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

According to some embodiments, a system and a method of Dynamic EMR Storage, Processing and Recommendation is disclosed.

Disclosed is a system, method and program product for storing, processing, analyzing and reporting patient records for endocannabinoid healthcare. An infrastructure is described that includes a system for submitting a query: a matching engine for matching the query against patient metadata obtained from a plurality of customer relation management (CRM and electronic medical record/electronic health record/electronic wellness record (EMR/EHR/EWR) systems to identify matching patients; a system for requesting applicable EMR/EHR/EWR systems to release patient details of a set of matching patients; a cohort data repository for collecting patient details from the applicable EMR/EHR/EWR systems, and a market place for the recommendation of treatments,

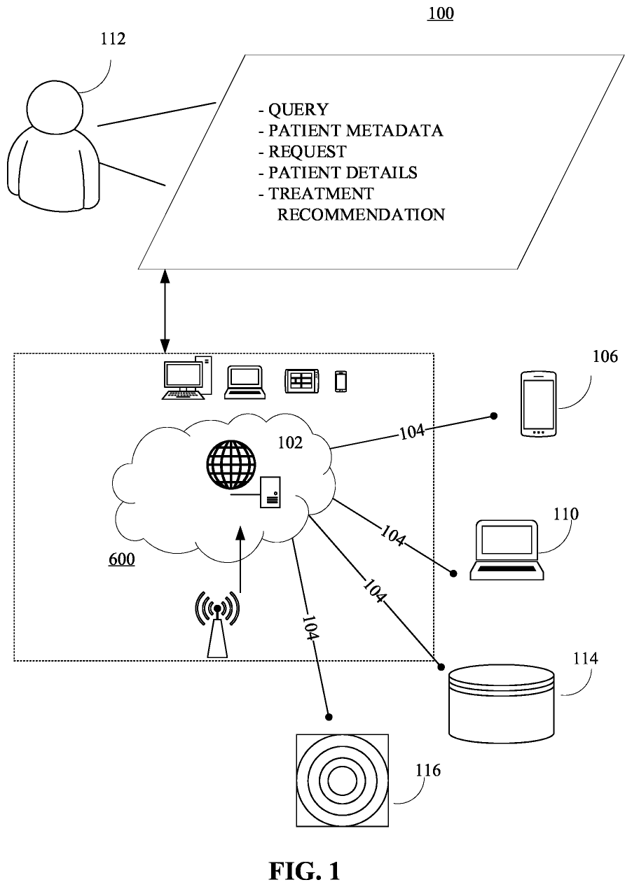

Referring now to figures, FIG. 1 is an illustration of an online platform 100 consistent with various embodiments of the present disclosure. By way of non-limiting example, the online platform 100 to facilitate management of patient records may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers, patients, healthcare professionals, care givers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 600.

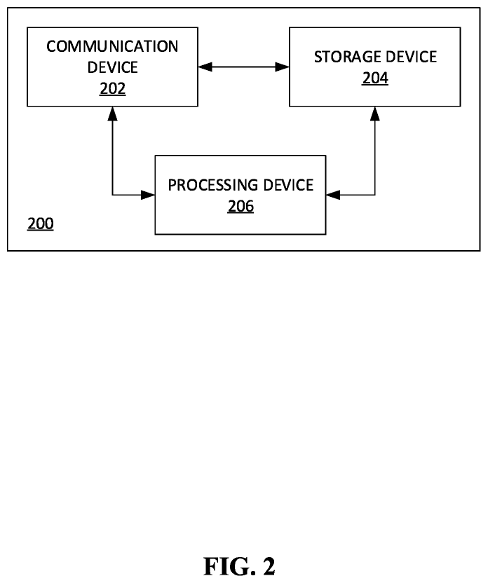

FIG. 2 is a block diagram of a system 200 for facilitating management of patient records, in accordance with some embodiments.

Further, the system 200 may include a communication device 202, a storage device 204, and a processing device 206. Further, the communication device 202 may be configured for receiving a query from a user device. In some embodiments, the query may include physiological data corresponding to a user of the user device. In some embodiments, the user device may include a wearable device.

Further, the communication device 202 may be configured for receiving a plurality of patient metadata from at least one patient metadata source. Further, the at least one patient metadata source may include at least one of one or more Customer Relation Management (CRM) systems and one or more Electronic Medical Record (EMR) systems.

Further, the communication device 202 may be configured for transmitting at least one request to at least one patient data source. Further, the at least one request may include indication of at least one matching patient.

In some embodiments, the at least one patient data source may include at least one of a Customer Relation Management (CRM) system, an Electronic Medical Record (EMR) system, an Electronic Health Record (EHR) system and Electronic Wellness Record (EWR).

In some embodiments, the at least one patient data source may include at least one of a dispensary database, a prescription database, and a laboratory database.

Further, the communication device 202 may be configured for receiving one or more patient details corresponding to the at least one matching patient from the at least patient data source. In some embodiments, the one or more patient details may be stored in a cohort data repository.

Further, the communication device 202 may be configured for transmitting at least one treatment recommendation to the user device. In some embodiments, the at least one treatment recommendation may correspond to endocannabinoid healthcare

Further, the storage device 204 may be communicatively coupled to the communication device 202. Further, the storage device 204 may be configured for storing the one or more patient details.

Further, the processing device 206 may be communicatively coupled to each of the storage device 204 and the communication device 202. Further, the processing device 206 may be configured for comparing the query with the plurality of patient metadata.

Further, the processing device 206 may be configured for identifying the at least one matching patient based on the comparing.

Further, the processing device 206 may be configured for analyzing the one or more patient details, and/or or being able to compare patient details and treatment outcomes, in a way that includes by is not limited to genetic information, demographic information and any other biologically relevant information and comparative data.

Further, the processing device 206 may be configured for generating the at least one treatment recommendation based on the analyzing.

Further, the processing device 206 may be configured for generating a reformulation for at least one treatment recommendation based on the analyzing, which may include but not limited to the patient in question and their data, linked related other patient data, historical and/or real-time data from clinical outcomes within the ecosystem.

In further embodiments, the communication device 202 may be configured for transmitting a prompt to the user device. Further, the query may be received in response to the prompt. Further, the processing device 206 may be configured for instantiating a chat-bot configured for generating the prompt.

In some embodiments, each of the analyzing of the one or more patient details and the generating of the at least one treatment recommendation may be performed based on machine learning.

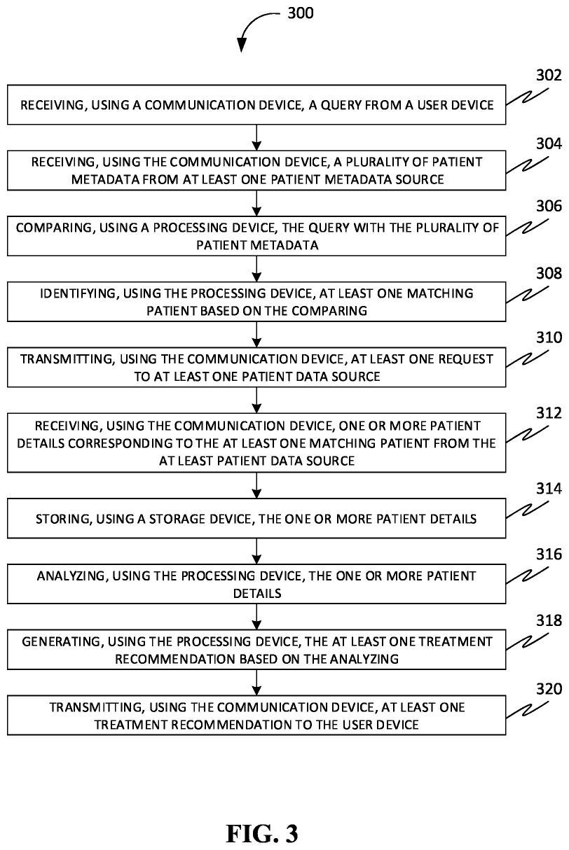

FIG. 3 is a flowchart of a method 300 for facilitating management of patient records, in accordance with some embodiments.

At 302, the method 300 may include receiving, using a communication device (such as the communication device 202), a query from a user device. In some embodiments, the query may include physiological data corresponding to a user of the user device. Further, in some embodiments, the user device may include a wearable device.

Further, at 304, the method 300 may include receiving, using the communication device, a plurality of patient metadata from at least one patient metadata source. In some embodiments, the at least one patient metadata source may include at least one of one or more Customer Relation Management (CRM) systems and one or more Electronic Medical Record (EMR) systems.

Further, at 306, the method 300 may include comparing, using a processing device (such as the processing device 206), the query with the plurality of patient metadata.

Further, at 308, the method 300 may include identifying, using the processing device, at least one matching patient based on the comparing.

Further, at 310, the method 300 may include transmitting, using the communication device, at least one request to at least one patient data source. Further, the at least one request may include indication of the at least one matching patient, or a match against a biological process, or an observed clinical outcome within the ecosystem.

In some embodiments, the at least one patient data source may include at least one of a Customer Relation Management (CRM) system, an Electronic Medical Record (EMR) system, an Electronic Health Record (EHR) system, and/or an Electronic Wellness Record (EWR).

In some embodiments, the at least one patient data source may include at least one of a dispensary database, a prescription database, and a laboratory database.

Further, at 312, the method 300 may include receiving, using the communication device, one or more patient details corresponding to the at least one matching patient from the at least patient data source. In some embodiments, the one or more patient details may be stored in a cohort data repository.

Further, at 314, the method 300 may include storing, using a storage device (such as the storage device 204), the one or more patient details.

Further, at 316, the method 300 may include analyzing, using the processing device, the one or more patient details.

Further, at 318, the method 300 may include generating, using the processing device, the at least one treatment recommendation based on the analyzing. In some embodiments, the at least one treatment recommendation may correspond to endocannabinoid healthcare.

Further, at 320, the method 300 may include transmitting, using the communication device, at least one treatment recommendation to the user device.

In some embodiments, each of the analyzing of the one or more patient details and generating of the at least one treatment recommendation may be performed based on machine learning.

This machine learning process enables the synthesis of existing formulations, reformulation of existing formulations for targeted patient treatment, and/or novel formulations.

In some embodiments, at least one treatment recommendation corresponds to endocannabinoid healthcare, psilocybin healthcare methylenedioxy-methylamphetamine healthcare, and its related derivatives, least one treatment recommendation corresponds to integrative healthcare, which may include by not limited to, yoga practice, which may include by not limited to, existing FDA approved drugs.

This is novel in helping to facilitate the secure means of tracking, administering and refining care, and includes but is not limited to discovering, identifying, administrating and/or refining for treatments where government regulatory frameworks may not be fully defined. This security is provided by either standard existing methods or blockchain technology to create a closed-loop ecosystem, where the data supply chain between physical goods and data are integrated, in what may be a feedback loop. The invention can also certify different levels of wellness care, across the entire ecosystem of health, wellness and/or integrative care.

The system generates the at least one treatment recommendation based on the analyzing,
generating new potential options for treatment reformulation, through either manual analysis or automated processing,
automatically provide necessary feedback to each part of the ecosystem supply chain, modularly,
the connectivity of the system and its communication is synchronous or asynchronous,

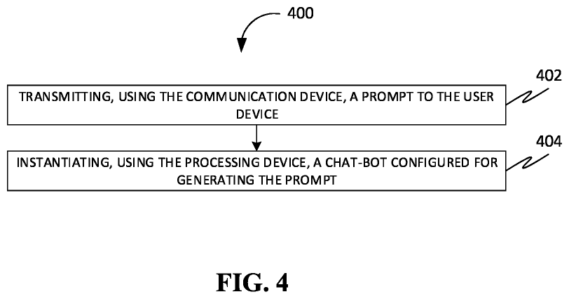

FIG. 4 is a flowchart of a method 400 for facilitating management of patient records, in accordance with further embodiments.

Further, at 402, the method 400 may include transmitting, using the communication device, a prompt to the user device. Further, the query may be received in response to the prompt.

Further, at 404, the method 400 may include instantiating, using the processing device, a chat-bot configured for generating the prompt.

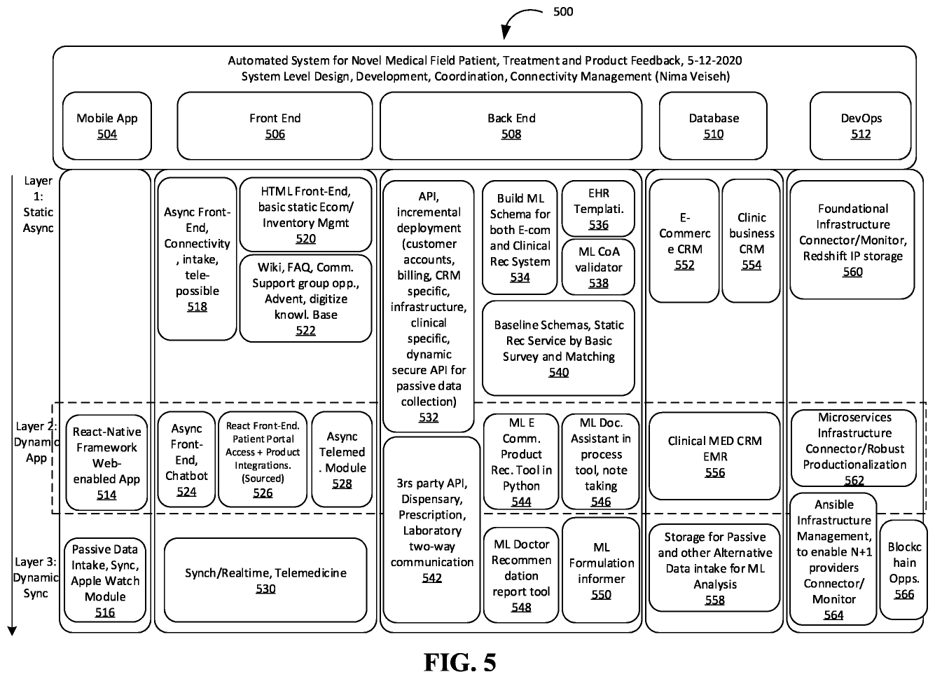

FIG. 5 is a block diagram of a system 500 for analyzing and reporting patient records for endocannabinoid healthcare, in accordance with some embodiments.

The system 500 may include one or more of a mobile application 504, a front end application 506, a back end application 508, a database 510, and DevOps 512.

The mobile application 504 may include a react-native framework web-enabled application 514 in a layer 2 (dynamic application). Further, the mobile application 504 may include a passive data intake, sync, apple watch module 516 in a layer 3 (dynamic sync).

Further, the front end application 506 may include an Async front-end, connectivity, intake, tele-possible 518, an HTML Front-End, basic static Ecom/inventory management 520 and a Wiki, FAQ, Comm. Support group opp., Advent, digitize knowledge base 522 in a layer 1 (static async).

Further, the front end application 506 may include an Async Front-End, Chatbot 524, React Front-End. Patient Portal Access+Product Integrations. (Sourced) 526 and an Async Telemedicine Module 528 in the layer 2 (dynamic application).

Further, the front end application 506 may include a Synch/Real-time, Telemedicine 530 in the layer 3 (dynamic sync).

Further, the back end application 508 may include an API, incremental deployment (customer accounts, billing, CRM specific, infrastructure, clinical specific, dynamic secure API for passive data collection, which may be, but not limited to, being facilitated by block chain technology) 532, a Build ML Schema for both E-commerce and Clinical Rec System 534, an EHR Templati 536, an ML CoA validator 538, Baseline Schemas, a Static Rec Service by Basic Survey and Matching 540, 3rd party API, Dispensary, Prescription, Laboratory two-way communication 542, an ML E Communication Product Rec. Tool in Python 544, an ML Doc. Assistant in process tool, note taking 546, an ML Doctor Recommendation report tool 548 and an ML Formulation informer 550.

Further, the database 510 may include an E-Commerce CRM 552, a Clinic business CRM 554, a Clinical MED CRM EMR 556 and a storage for Passive and other Alternative Data intake for ML Analysis 558.

Further, the DevOps 512 may include a Foundational Infrastructure Connector/Monitor, a Redshift IP storage 560, a Micro-Services Infrastructure Connector/Robust Productionalization 562, an Ansible Infrastructure Management, to enable N+1 providers Connector/Monitor 564 and Blockchain Ops. 566.

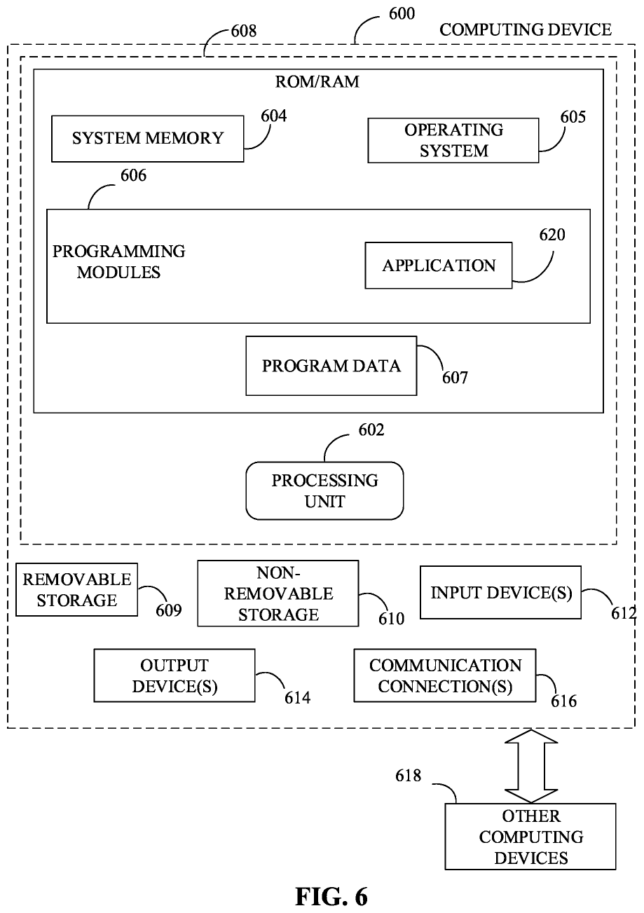

With reference to FIG. 6, a system consistent with an embodiment of the disclosure may include a computing device or cloud service, such as computing device 600. In a basic configuration, computing device 600 may include at least one processing unit 602 and a system memory 604. Depending on the configuration and type of computing device, system memory 604 may comprise, but is not limited to, volatile (e.g. random-access memory (RAM)), non-volatile (e.g. read-only memory (ROM)), flash memory, or any combination. System memory 604 may include operating system 605, one or more programming modules 606, and may include a program data 607. Operating system 605, for example, may be suitable for controlling computing device 600's operation. In one embodiment, programming modules 606 may include customer relation management (CRM) module, electronic medical record/electronic health record (EMR/EHR) module. Furthermore, embodiments of the disclosure may be practiced in conjunction with a graphics library, other operating systems, or any other application program and is not limited to any particular application or system. This basic configuration is illustrated in FIG. 6 by those components within a dashed line 608.

Computing device 600 may have additional features or functionality. For example, computing device 600 may also include additional data storage devices (removable and/or non-removable) such as, for example, magnetic disks, optical disks, or tape. Such additional storage is illustrated in FIG. 6 by a removable storage 609 and a non-removable storage 610. Computer storage media may include volatile and non-volatile, removable and non-removable media implemented in any method or technology for storage of information, such as computer-readable instructions, data structures, program modules, or other data. System memory 604, removable storage 609, and non-removable storage 610 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 600. Any such computer storage media may be part of device 600. Computing device 600 may also have input device(s) 612 such as a keyboard, a mouse, a pen, a sound input device, a touch input device, a location sensor, a camera, a biometric sensor, etc. Output device(s) 614 such as a display, speakers, a printer, etc. may also be included. The aforementioned devices are examples and others may be used.

Computing device 600 may also contain a communication connection 616 that may allow device 600 to communicate with other computing devices 618, such as over a network in a distributed computing environment, for example, an intranet or the Internet. Communication connection 616 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 604, including operating system 605. While executing on processing unit 602, programming modules 606 (e.g., application 620) may perform processes including, for example, one or more stages of methods, algorithms, systems, applications, servers, databases as described above. The aforementioned process is an example, and processing unit 602 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A system for facilitating management of patient records, the system comprising:
a communication device configured for:
receiving a query from a user device;
receiving a plurality of patient metadata from at least one patient metadata source;
transmitting at least one request to at least one patient data source, wherein the at least one request comprises indication of at least one matching patient;
receiving one or more patient details corresponding to the at least one matching patient from the at least patient data source; and
transmitting at least one treatment recommendation to the user device;
a storage device communicatively coupled to the communication device, wherein the storage device is configured for storing the one or more patient details; and
a processing device communicatively coupled to each of the storage device and the communication device, wherein the processing device is configured for:
comparing the query with the plurality of patient metadata;
identifying the at least one matching patient based on the comparing;
analyzing the one or more patient details; and
generating the at least one treatment recommendation based on the analyzing.

2. The system of claim 1, wherein the at least one patient metadata source comprises at least one of one or more Customer Relation Management (CRM) systems and one or more Electronic Medical Record (EMR) systems.

3. The system of claim 1, wherein the at least one patient data source comprises at least one of a Customer Relation Management (CRM) system, an Electronic Medical Record (EMR) system and an Electronic Health Record (EHR) system.

4. The system of claim 1, wherein the at least one patient data source comprises at least one of a dispensary database, a prescription database, and a laboratory database.

5. The system of claim 1, wherein the one or more patient details are stored in a cohort data repository.

6. The system of claim 1, wherein the at least one treatment recommendation corresponds to endocannabinoid healthcare.

7. The system of claim 1, wherein the query comprises physiological data corresponding to a user of the user device.

8. The system of claim 1, wherein the user device comprises a wearable device.

9. The system of claim 1, wherein the communication device is further configured for transmitting a prompt to the user device, wherein the query is received in response to the prompt, wherein the processing device is configured for instantiating a chat-bot configured for generating the prompt.

10. The system of claim 1, wherein each of the analyzing of the one or more patient details and the generating of the at least one treatment recommendation is performed based on machine learning.

11. A method for facilitating management of patient records, the method comprising:
receiving, using a communication device, a query from a user device;
receiving, using the communication device, a plurality of patient metadata from at least one patient metadata source;
comparing, using a processing device, the query with the plurality of patient metadata;
identifying, using the processing device, at least one matching patient based on the comparing;
transmitting, using the communication device, at least one request to at least one patient data source, wherein the at least one request comprises indication of the at least one matching patient;
receiving, using the communication device, one or more patient details corresponding to the at least one matching patient from the at least patient data source;
storing, using a storage device, the one or more patient details;
analyzing, using the processing device, the one or more patient details;
generating, using the processing device, at least one treatment recommendation based on the analyzing; and
transmitting, using the communication device, the at least one treatment recommendation to the user device.

12. The method of claim 11, wherein the at least one patient metadata source comprises at least one of one or more Customer Relation Management (CRM) systems and one or more Electronic Medical Record (EMR) systems.

13. The method of claim 11, wherein the at least one patient data source comprises at least one of a Customer Relation Management (CRM) system, an Electronic Medical Record (EMR) system and an Electronic Health Record (EHR) system.

14. The method of claim 11, wherein the at least one patient data source comprises at least one of a dispensary database, a prescription database, and a laboratory database.

15. The method of claim 11, wherein the one or more patient details are stored in a cohort data repository.

16. The method of claim 11, wherein the at least one treatment recommendation corresponds to endocannabinoid healthcare.

17. The method of claim 11, wherein the query comprises physiological data corresponding to a user of the user device.

18. The method of claim 11, wherein the user device comprises a wearable device.

19. The method of claim 11 further comprising:
transmitting, using the communication device, a prompt to the user device, wherein the query is received in response to the prompt; and
instantiating, using the processing device, a chat-bot configured for generating the prompt.

20. The method of claim 11, wherein each of the analyzing of the one or more patient details and the generating of the at least one treatment recommendation is performed based on machine learning.
